(19) United States
(12) Patent Application Publication
(10) Pub. No.: US 2009/0286263 A1
(43) Pub. Date: Nov. 19, 2009

(54) SKIN TAPE STRIPPING: A NON-INVASIVE DIAGNOSTIC STRATEGY FOR DERMAL EXPOSURE TO CYTOTOXIC COMPOUNDS

(75) Inventors: John S. Graham, Bel Air, MD (US); John P. Petrali, Churchville, MD (US)

Correspondence Address: United States Army Medical Research;and Material Command MCMR-ZA-J, 504 Scott Street, Fort Detrick, MD, 21702-5012, US

(73) Assignee: United States Army as represented by the Secretary of the Army, Fort Detrick, MD (US)

(21) Appl. No.: 12/398,010

(22) Filed: Mar. 4, 2009

Related U.S. Application Data
(60) Provisional application No. 61/070,619, filed on Mar. 5, 2008.

(51) Int. Cl. G01N 33/567; C07K 16/00

(52) U.S. Cl. 435/7.21; 530/389.8

(57) ABSTRACT

A non-invasive method for determining a subject's dermal exposure to an agent employing an immunohistochemical procedure on a skin strip applied to an exposed area on the subject's person and detecting agent adduct thereon.

### CROSS-REFERENCE TO RELATED APPLICATION

This application claims the benefit of Provisional Application No. 61/070,619, filed Mar. 5, 2008; the contents of which are hereby incorporated by reference.

### RIGHTS

This invention was made with support from the United States Government, specifically, the United States Army Medical Research and Materiel Command, and, accordingly, the United States has certain rights in this invention.

### FIELD AND BACKGROUND

Mustard agents are vesicants (blister agents) used in warfare to produce casualties, degrade fighting efficiency, and force opposing troops to wear full protective equipment. Mustard agents are cytotoxic alkylating compounds and include nitrogen mustards (HN-1, HN-2, HN-3), sulfur mustards (H, HD, HT), and mustard-lewisite (HL) (abbreviations are NATO designations according to purity grades). Mustard agents are oily liquids ranging from colorless (in pure state) to pale yellow to dark brown, depending on the type and purity. They have a faint odor of mustard, onion, garlic, or horseradish. Volatility varies with the particular compound.

More than 2 dozen nations may have the capability to manufacture offensive chemical weapons. Mustard agents are simple to manufacture and therefore can be a first choice for countries or terrorists who decide to have a capacity for chemical warfare agents. Mustard agents may be delivered by artillery shell, mortar shell, rockets, bombs, or aircraft spray. Since World War I, mustard use in at least 12 conflicts has been supported by evidence or allegations. Historically, mustard agents are the most widely used type of chemical warfare agent.

Mustard agents constitute both a vapor and a liquid threat. Mustard agents cause tissue damage within several minutes of contact. No immediate symptomatic or local reaction occurs to mustard vapor or liquid. Decontamination must be performed immediately after contact to prevent injury. A latent period occurs, ranging from 4-12 hours after mild exposure and 1-3 hours after severe exposure, prior to the onset of symptoms. More than 80% of mustard casualties are from vapor exposure, but more severe injuries are caused after contact with liquid mustard agents.

Developing nations and terrorist groups can easily obtain HD because of its low cost and availability. The US stockpile of mustard chemical warfare agents currently is undergoing destruction.

### Pathophysiology

Mustard agents are lipophilic and are absorbed readily across intact skin and mucous membranes. The rapid penetration is enhanced by moisture, heat, and thin skin. The physical properties (low volatility and a freezing point of 14 degrees centigrade) of sulfur mustard (H, HD, HT) make it a better weapon for use in warm or hot environments due to a greater risk of vapor inhalation. Approximately 20% of HD is absorbed by the skin, the remainder evaporates. Of the absorbed HD, 10-50% of the mustard dose binds to the skin as reacted (fixed) mustard, and the remaining 50-90% is distributed in the circulation as unreacted (free) mustard to almost all organs and tissues. Because of dilutional effects, systemic effects are observed only at high doses. Mustard is eliminated from the body in the urine as a by-product of alkylation.

The threshold amount of mustard vapor for a skin lesion (erythema) is generally 200 mg*min/m³, although the exact amount varies depending on changes in physiological conditions (including sweat and skin pigmentation) and the environment.

A liquid droplet of about 10 μg will cause vesication.

No single mechanism or clear understanding exists for the biological damage caused by mustard agents. The toxic effects of mustards depend on their rapid covalent binding to a large number of nucleophilic biological molecules and in the formation of a reactive cyclic ethylene sulfonium ion. Mustard agent molecules contain 2 reactive binding groups. Mustards can bind to nucleophiles such as nitrogen in the base components of nucleic acids and sulfur in SH-groups in proteins and peptides. Mustards can destroy a large number of cellular substances by alkylation of DNA, which leads to DNA strand breaks and apoptosis. The products of these reactions are stable adducts that can modify the normal function of the larger macromolecule. Nucleophilic areas exist in peptides, proteins, RNA, DNA and membrane components of living tissues. These adducts, are therefore, extensive after exposure.

Mustards also bind to cellular glutathione, a small peptide that is a major free radical scavenger. Glutathione depletion leads to inactivation of enzymes, loss of calcium homeostasis, lipid peroxidation, cellular membrane breakdown, and cell death. Pretreatment of cells with N-acetylcysteine has shown benefit in some studies.

For a detailed over of the proposed biochemical mechanisms of mustard toxicity see Sidell et al., “Sulfur Mustard: A Chemical Vesicant Model”, Chapter 9, pp. 119-127 (5th Ed.) [1] the contents of which are incorporated by reference herein in their entirety.

Individual cell death within 2 hours of vapor exposure has been demonstrated in an animal model and general cell necrosis within 12 hours. During the first 24 hours the pathology involves the latent-lethal targeting of epidermal basal cells, a disabling of hemidesmosomes, and a recruitment of inflammatory cells within the dermal vasculature, all beginning 4-6 hours post exposure (prevesication), following with an onset of progressive inflammatory edema of the lamina lucida contributing to the formation of characteristic lucidolytic microvesicles persisting at the dermal-epidermal junction (vesication).

During prevesication, subcellular effects of sulfur mustard exposure include condensation and margination of nuclear chromatin, dilatations of the nuclear envelope, mitochondrial swelling, disabling of desmosomes and hemidesmosomes, tonofilament condensations, widening of intercellular spaces, and plasmalemmal defects.

Vesication is characterized by extensive paranuclear and cytoplasmic vacuolations, swollen endoplasmic reticulum, mitochondrial densities, nuclear pyknosis, cellular fragmentation, and necrosis involving suprabasal cells and cell of the stratum spinosum. Microvesicles form within the lamina lucida of the basement membrane and become rapidly inflitratred with inflammatory cells, phagocytic cells, degenerating cells, cellular debris and tissue fluid form pervasive lucidolytic microblisters which cleave the epidermis from the dermis.

### Mortality/Morbidity

The concentration-time product capable of killing 50% of exposures (LCt50) of mustard vapor is 1500 mg·min/m3, and the lethal dose to 50% of exposures (LD50) of liquid mustard on the skin is 100 mg/kg.

### Diagnosis

No hospital laboratory test exists to identify or quantify mustard exposures, since mustard is biotransformed and bound to tissues within minutes of adsorption.

Leukocytosis occurs during the first day. After large systemic adsorption, leukopenia may begin on day 3-5. A leukocyte count of 500 or less is an unfavorable prognostic sign.

The US military has the capability of detecting mustard agents in the environment with the use of the M256A1, M272 water testing kit, miniature chemical agent monitor (MINICAMS), individual chemical agent detector (ICAD), M18A2, M21 remote sensing alarm, M90, M93A1 Fox, Bubbler, chemical agent monitor (CAM), depot area air monitoring system (DAAMS), and M8 or M9 chemical detection paper.

### Treatment

Unless carried out within 1-2 minutes, decontamination of victims exposed to mustard agents does not prevent subsequent blistering. After that brief window, decontamination still should be carried out to prevent secondary contamination.

Exposed skin and scalp can be decontaminated using the military M291 or M258A1 skin decontamination kits. Alternately, use 0.5% aqueous chlorine solution to thoroughly wash the skin and hair. Wash off the decontamination solutions within 3-4 minutes with soap and water. If the victim already has erythematous skin, decontaminating the skin with just soap and water is recommended.

No specific treatment or antidote can reverse or prevent the cellular effects of mustard agents.

The pathology after sulfur mustard (HD) exposure occurs in two phases. It is initially quiescent and then becomes blister forming. HD-induced skin pathology occurs during the quiescent prevesication phase and is characteristic of the vesication stage as well. Prevesication (up to approximately 9 hours post HD exposure) is characterized by site erythema while the vesication phase is recognized by the formation of characteristic microblisters at the epidermal-dermal junction which at later periods coalesce to begin the process of cleavage of the epidermis from the dermis. This process which occurs in animal studies can be extrapolated to the pathogenesis of the typical human response of characteristic surface fluid-filled bullae to sulfur mustard skin exposures.

Because of the speed in which HD binds to the skin it is important that the exposure to HD be diagnosed in its earliest stages because the immediate removal of the agent from the skin is the only effective means of preventing or decreasing tissue damage [1]. At this time, there is no definitive diagnostic strategy for determining exposure to HD. Any useful diagnostic strategy would have to be practiced during the clinically quiescent prevesication phase, before the onset of the characteristic bullae formations.

Persistent immunopathological injuries that occur during prevesication are alkylations of epidermal structural proteins. Keratins, an abundant structural resident protein of the epidermis, when exposed to the alkylating effects of HD, undergo specific conformational changes of selected amino acids (i.e., glutamines, cysteines, asparagines) which result in the formation of HD adducts of keratin. Specific antibody for keratin adducts have now become available for immunohistochemical detection and visualization of adducted keratin sites. Other epidermal cells, when exposed to HD, have been found by applicants to form adducts with HD as well. Applicants have developed antibodies useful for detecting global pathology to all HD cellular adducts formed.

Detection of biological agents currently relies upon nucleic acid and immunological based methodologies. Immunological assays rely on antibodies for binding and identifying different targets. Antibodies are excellent reagents as they are able to bind biological agents with high affinity and specificity. Several different assay platforms exist, and their widespread use will require high quality antibodies in sufficient quantities to maintain or improve existing detection capabilities [2].

Current immunological assays capable of detecting many biological agents utilize the sensitivity and specificity of polyclonal and monoclonal antibodies. Polyclonal antibodies (pAb) are produced by immunization of a host animal such as a rabbit or goat. Immunoglobulins are purified from the sera and used for bio-detection. Each host animal responds uniquely to a given immunization protocol, requiring validation of each lot of purified pAb and re-optimization of immunological assays. A finite supply of anti-serum can be produced from a given host animal which necessitates an ongoing effort to produce a wide array of anti-sera to supply deployed biological agent detectors [2].

Monoclonal antibody (mAb) technology takes a single antibody-producing B cell from a mouse and immortalizes it by fusion with a myeloma cell line, creating a hybridoma cell line. Monoclonal antibodies are preferred for use on immunosensors because of the specificity and standardization afforded among labs using the same cell line. While monoclonal antibodies produced by mammalian hybridoma lines are consistent in their recognition of a single epitope, media and serum costs can make them expensive to produce. In addition, hybridoma cells only produce murine antibodies and they require specific maintenance procedures for long-term storage. When performing hybridoma fusions it can be difficult to isolate specific clones that recognize a unique epitope on a bio-threat agent when that marker is rare, sterically shielded, or not immunodominant [2].

Recombinant antibodies are similar to mAbs but consist of only the antigen binding domains and are produced from immune tissue or hybridoma cell lines through the use of recombinant DNA technology. Unlike traditional pAbs and mAbs, recombinant antibodies are maintained in bacteria, offer a stable genetic source, and can be genetically manipulated. The ability to produce a large number of recombinant antibodies in bacterial cells and to select for antibodies that bind to unique or non-dominant epitopes demonstrates the power of a recombinant approach to antibody development. Expression and purification of recombinant antibodies by bacterial fermentation is less expensive, easier to perform, and less time consuming than production of either pAbs or mAbs [2].

Phage displays and phage antibodies have recently grown in prominence in the area of biomonitoring and chemical defense [3, 4]. Antibody genes are pooled together to create a combinatorial library of antibodies that are introduced into Escherichia coli bacteria by electroporation. The bacteria are then co-infected with a helper bacteriophage virus that causes each unique bacterial clone to produce thousands of progeny virus which display its unique Fab antibody on the surface of the virus as a fusion with a normally occurring coat protein. The display of the antibody on the bacterial virus allows for a selection process called biopanning [5] in which the viruses are passed over a solid support to which the target of interest is immobilized. Virus which display a Fab that recognizes the target bind to the support, while those displaying antibodies that do not bind to the target antigen are washed away with buffer. The enriched bound fraction is eluted with acid and amplified for another round of panning. Repeating the process through successive rounds can result in an increase from 0.005 to >75% antigen binding clones [6].

Despite its promise, however, phage-antibody technology is not without difficulties. The last step in particular—expressing the selected antibody genes to make usable quantities of antibody—has proven troublesome, differing idiosyncratically from one antibody to another [2]. Recombinant antibodies are sensitive to many stresses, so their engineered modification may be required to enhance their stability in nonphysiological conditions [7]. These considerations have lead to the development of non-immunoglobulin scaffolds to serve as the framework for artificial antibodies [8].

Several classes of protein scaffolds proved to yield reagents with specificities and affinities in a range that of antibodies. These scaffolds are obtained by designing a random library with mutagenesis focused at a loop region or at an otherwise permissible surface area and by selection of variants against a given target via phage display or related techniques. Only a few non-immunoglobulin scaffolds offer practical benefits such as robustness, smaller size, and ease of expression that justify their use as a true alternative to conventional antibodies or their recombinant fragments [9].

Currently, the most promising scaffolds with broader applicability are protein A, the lipocalins, a fibronectin domain, an ankyrin consensus repeat domain, and thioredoxin [9].

The application of immunoassay techniques such as ELISA is well established and can fulfill a vital role in detecting and quantitating biological materials and toxins. This assay format is easily implemented in the laboratory using processed samples, but the challenge is to develop immunoassays that will detect biological agents in the environment. Such assays must be specific, adaptable to field conditions, easy to use, inexpensive, and sensitive. Ideally, they would also be stable at room temperature for extended periods of time to facilitate stockpiling or shipping [2].

Environmental monitoring of biological material in the field can be qualitatively analyzed by using immunochromatographic hand-held assay similar in principle to commercially available home pregnancy test kits. These single step assays require little development time, can be quickly assembled using commercially available resources, and require no additional reagents. A drawback of this approach is that quantitative assessments based on color development are subjective unless corrected for time, and analyzed by an automated color densitometer. Furthermore, these assays are not always as sensitive as other more cumbersome multi-step assays. However, hand-held assays can fill an important niche for the military and civilian first responders. A rapid yes or no assessment is useful prior to initiating an area sampling strategy or to order the donning of protective garments. Chromatographic assays have the advantage of portability, stability, and ease of use that other antibody-based protocols lack [2].

One such hand-held chromatographic assay is described by Emanuel et al. [2]. The system, as described, consists of two separately placed antibody capture lines striped on a nitrocellulose membrane, one consisting of the target antibody and the other providing a goat anti-mouse control antibody. A positive sample is indicated if both lines turn purple within 15 minutes. However, this kit has some serious drawbacks including a lack of stability of the recombinant Fabs utilized, a lack of sensitivity, a short shelf-live and the need for an automated color densitometer [2]. A better, field expedient chromatographic immunoassay that addresses these problems is, therefore, needed.

Most of the currently available methods for the determination of sulfur mustard exposure in urine [10-13], blood [10, 14-18], and skin [10, 14, 15, 17] are mainly based on mass spectrometric and immunochemical detection of metabolites and adducts on DNA and proteins. Phage display has also proven to be a useful method of isolation of antibodies recognizing a broad range of antigens [2, 3, 19, 20].

Another known approach to investigate physical, chemical, or structural properties of the stratum corneum as well as the penetration of the stratum corneum (absorption) by chemical compounds employed physical removal of the surface cell layers by tape stripping [21-28]. These studies offer a starting point for development of techniques and methods useful for measuring dermal exposure in occupational field studies. However, these studies did not allow for investigation of dermal exposure per se since the contaminant was washed from the skin prior to the application of the tape.

Although promising, tape stripping of the stratum corneum for estimation of dermal exposure has been used only with a few compounds of limited interest for occupational health. Cullander et al. [29, 30] described a tape-stripping method for the analysis of metals in the skin. However these techniques require the use of specially designed low-metal content tape suitable for use with particle-induced X-ray emission (PIXE), an analytical technique based on X-ray spectrometry.

Although skin tape peels have been used in dermatological practice and specific investigative study for some time [31], the analysis of samples is typically processed and analyzed in a laboratory using methods such as gas chromatography not readily available in remote locations and requiring heavy use of limited resources and expertise.

Skin tape stripping has been practiced in dermatology to measure the physical/chemical condition of skin remaining on a subject after stripping and for qualitative analysis of exogenous or endogenous compounds present within the skin. The tape used for stripping and the cells present thereon were discarded. Use of the skin cells that adhere to the tape that is employed has, heretofore, not been used to determine whether exposure to HD has occurred.

Although the practice of skin tape stripping has also been used investigatively to measure the presence of some skin irritants that adhere to the tape and that remain on the skin after tape stripping, such as surface wood coatings, it has yet to be used as a non-invasive diagnostic strategy for skin exposure to chemical and biological agents.

A description of the type of antibodies specific to keratin adducts suitable for use in detecting exposure to sulfur mustard is set forth in van der Schans et al., “Immunochemical Detection of Sulfur Mustard Adducts with Keratins in the Stratum Corneum of Human Skin,” Chem. Res. Toxicol., 2002, 10, 21-25 [15]. However, the protocols described in this reference require the preparation of skin cryostat sections and immunofluorescence microscopy requiring controlled temperatures, a 24 hour-plus timeline and a laser scanning microscope. Such a protocol is not only highly time and resource intensive but not conducive to the field expedient requirements that typify the scenario where exposure to sulfur mustard is most common (for example, in an emergency urban setting, or on the battlefield).

Information relevant to attempts to address these problems can be found in U.S. Pat. No. 5,088,502 to Miller and U.S. patent application Ser. No. 11/710,661 to Benson. However, each of these references suffer from one or more of the following disadvantages: in Benson—the processing of RNA and/or DNA nucleotides in a controlled environment requiring sophisticated laboratory instruments to determine indentify and quantity (RNEASY™ Qiagen, Valencia, Calif. and TRIREAGENT™, Molecular Research Center, Inc., Cincinnati, Ohio); the use of biochips and similar microarrays and conditions necessary to allow for hybridization reactions; and, in Miller—the design is specific to visual inspection in normal light environments and not conducive to immunohistochemical processing and microscopic examination.

Therefore a need exists for a non-invasive, field expedient strategy that is quickly confirmatory for sulfur mustard skin exposures.

Furthermore, a need exists for a procedure applicable for use as a field testing kit.

### BRIEF DESCRIPTION

A non-invasive method for determining a subject's dermal exposure to an agent, wherein said method comprises: applying a transparent adhesive tape to a target area of the skin of the subject in a manner sufficient to isolate an epidermal sample adhering to the adhesive tape, wherein the epidermal sample comprises cells from the stratum corneum of the subject; employing an immunohistochemical procedure on the sample present on said tape resulting in a treated sample, said immunoshitochemical procedure employing an antibody specific to the adducts of said agent; and examining the treated sample present on said tape to determine the presence of said agent adduct therein.

One embodiment of the present invention is a method to non-invasively determine, during the quiescent phase of the pathology, whether HD exposure has occurred and a kit for accomplishing this method. In addition, applicants have developed a novel antibody for use in the method wherein this antibody specifically detects the global pathology present in cells following their exposure to HD. In the method herein, commercially available double-sided, transparent, sticky skin tape is attached to a slide and then the side of the tape not attached to the slide may be pressed onto the skin to be evaluated for HD exposure. The surface skin cells removed by the tape, and remaining adherent/present on the tape, are then subjected to routine immunohistochemical procedures followed by photomicroscopy to determine if exposure to HD has occurred.

The approach employed herein is to use dermal tape approved for medical use to strip superficial skin cells of the stratum corneum (upper most layer of the epidermis) of selected skin sites exposed to vesicating doses of HD vapor. Removed skin cells adhering to the stripping tape are processed with indirect immunoperoxidase procedures on-the-tape to visualize HD-adducted keratins.

The invention herein further teaches the use of dermal tape stripping of HD-exposed skin combined with specific immunohistochemical procedures to visualize all HD adducts formed—through use of antibodies having specificity to the global cytopathology induced by HD.

Human use is limited to in-vitro models of human skin explants recovered from corrective surgery, and bioengineered three-dimensional organotypic human skin. The in-vivo model of choice is the hairless guinea pig (HGP).

### BRIEF DESCRIPTION OF THE DRAWINGS

In the drawings:

FIG. 1 is a perspective view drawing of the constituent elements of a desmosome.

FIG. 2 is a cross-section drawing of the human epidermis.

FIG. 3 is a photograph of a typical appearance of stripped cells on tape A at a microscopic magnification 60×.

FIG. 4 is a photograph of stripped cells on tape Y without pretreatment. Methylene blue and azure II stain performed on hotplate and at a microscopic magnification 60×.

FIG. 5 is a photograph of stripped cells on tape Y with 1% Triton X-100 pretreatment. Methylene blue and azure II stain performed on hotplate and at a microscopic magnification of 60×.

FIG. 6 is a photograph of stripped cells on tape Z without pretreatment. Methylene blue and azure II stain performed on hotplate and at a microscopic magnification of 60×.

FIG. 7 is a photograph of stripped cells on tape Z with 100% methanol pretreatment. Methylene blue and azure II stain performed on hotplate and at a microscopic magnification of 60×.

FIG. 8 is a photograph of immunoreacted cells that have not been exposed to HD (control).

FIG. 9 is a photograph of HD-exposed cells 6 hours post exposure immunoreacted for HD adducts (positive stain—dark brown).

FIG. 10 is a photograph of HD-exposed cells 6 hours post exposure immunoreacted for HD adducts (positive stain—dark brown).

FIG. 11 is a photograph of illustrates HD exposed cells stained non-specifically with methylene blue for morphological identification.

### DETAILED DESCRIPTION OF THE EMBODIMENTS

The practice of the present invention will employ, unless otherwise indicated, conventional methods of chemistry, biochemistry, recombinant DNA techniques and immunology, within the skill of the art.

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to an antigen includes a mixture of two or more antigens, and the like.

The following amino acid abbreviations are used throughout the text:

Alanine: Ala (A) Arginine: Arg I Asparagine: Asn (N) Aspartic acid: Asp (D) Cysteine: Cys (C) Glutamine: Gln (Q) Glutamic acid; Glu (E) Glycine; Gly (G) Histidine: His (H) Isoleucine: Ile (I) Leucine: Leu (L) Lysine: Lys (K) Methionine: Met (M) Phenylalanine: Phe (F) Proline: Pro (P) Serine: Ser (S) Threonine: Thr (T) Tryptophan: Trp (W) Tyrosine: Tyr (Y) Valine: Val (V).

### Definitions

In describing the present invention, the following terms will be employed, and are intended to be defined as indicated below.

“Adduct” refers to a product of a direct addition of two or more distinct molecules, resulting in a single reaction product containing all atoms of all components, with formation of two chemical bonds and a net reduction in bond multiplicity in at least one of the reactants. A keratin adduct is the resultant product of the chemical reaction between sulfur mustard and skin.

As used herein, “antibodies” refers to tetramers or aggregates thereof which have specific immunoreactive activity, comprising light and heave chains usually aggregated in a “Y” configuration, with or without covalent linkage between them.

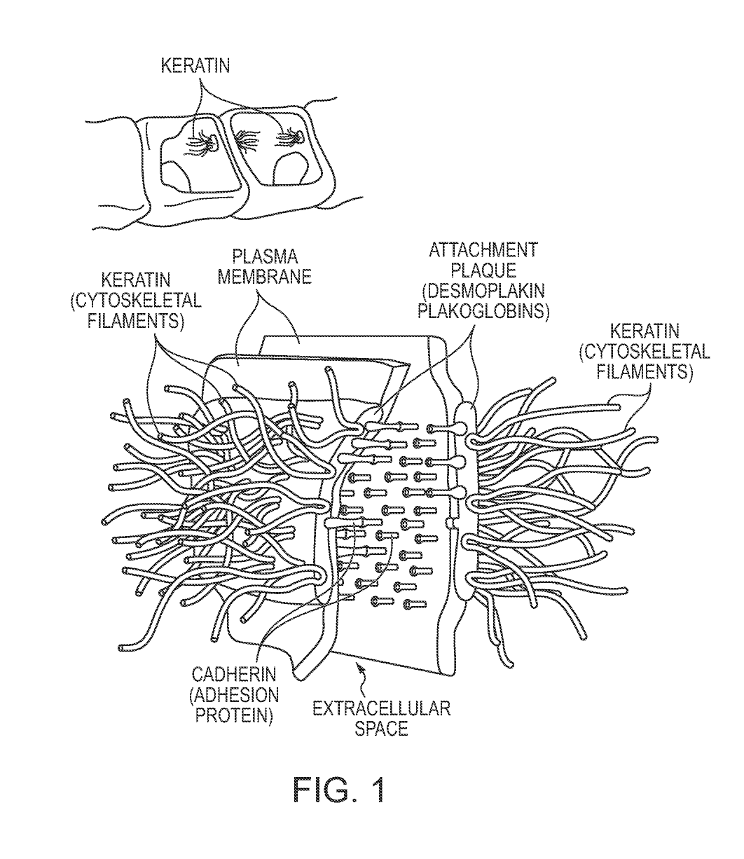

“Desmosomes” are molecular complexes of cell adhesion proteins and linking proteins that attach the cell surface adhesion proteins to intracellular keratin cytoskeletal filaments. See FIG. 1.

The “direct method of immunohistochemcial staining” is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The term, “Freund's adjuvants” includes both Freund's Complete Adjuvant (FCA) and Freund's Incomplete Adjuvant (FIA). FCA is a water-in-oil emulsion that localizes antigen for release periods up to 6 months. It is formulated with mineral oil, the surfactant mannide monoleate and heat killed Mycobacterium tuberculosis, Mycobacterium butyricum or their extracts (for aggregation of macrophages at the inoculation site). This adjuvant stimulates both cell mediated and humoral immunity with preferential induction of antibody against epitopes of denatured proteins. Freund's Incomplete Adjuvant has the same formulation as FCA but does not contain mycobacterium or its components. Freund's adjuvants are normally mixed with equal parts of antigen preparations to form stable emulsions.

“Hemidesmosomes” are similar to desmosomes except that, rather than linking two cells together, they function to attach a single cell to the extracellular matrix. Rather than using cadherins, hemidesmosomes use integrin cell adhesion proteins. Hemidesmosomes are asymmetrical and are found in epithelial cells connecting the basal face to other cells.

The “indirect method of immunohistochemical staining” uses one antibody against the antigen being probed for, and a second, antibody against the first (the secondary antibody must be raised against the IgG of the animal species in which the primary antibody has been raised.) This method is more sensitive due to signal amplification through several secondary antibody reactions with different antigenic sites on the primary antibody. The second layer antibody can be labeled with a fluorescent dye or an enzyme. In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining. The classical method for horseradish peroxidase (HRP) demonstration is the diaminobenzidine technique. Incubation of tissues containing HRP in a medium containing suitably buffered 3,3′-diaminobenzidine and H₂O₂ results in the deposition of a light-microscopically visible brown reaction product [32]. The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

The term “keratins” refers to a family of fibrous structural, insoluble proteins; that form the hard but nonmineralized structures found in reptiles, birds, amphibians and mammals. Keratins are a primary constituent protein of human skin, hair and nails.

A “monoclonal antibody” refers to homogenous populations of immunoglobulins derived from a single cell line.

A “polyclonal antibody” refers to immunoglobulins produced by immunization of a suitable mammal, such as a mouse, rabbit or goat. An antigen is injected into the mammal. This induces the B-lymphocytes to produce IgG immunoglobulins specific for the antigen. This polyclonal IgG is polyclonal purified from the mammal's serum.

As used herein, the term “tape” includes, illustratively, an acrylate or rubber based adhesive in a, for example, acrylic, polyethylene or polyester carrier (backing), and wherein the tape is pliable. The rubber based adhesive can be, for example, a synthetic rubber-based adhesive. The rubber based adhesive in illustrative examples, has high peel, high shear, and high tack. For example, the rubber based adhesive can have a peak force tack that is at least 25%, 50% or 100% greater than the peak force tack of an acrylic-based tape such as D-SQUAME™ (CuDerm Corp, Dallas, Tex.). The D-SQUAME™ tape has been found to have a peak force of 2 Newtons, wherein peak force of 2 Netwons, wherein peak force of the rubber based adhesive used for methods provided herein, can be 4 Newtons or greater. Furthermore, the rubber based adhesive can have adhesion of 0.0006 Newton meters, whereas the rubber based tape provide herein can have an adhesion of about 0.01 Newton meters using a texture analyzer. The rubber-based adhesive is on a support, carrier or backing, typically a film that makes the tape liable and flexible. In certain aspects, the tae can be soft and pliable. “Pliable” tape is tape that is easily bent or shaped. “Soft and pliable” tape is tape that is easily bent or shaped and yields readily to pressure or weight. The film can be made of any of many possible polymers, provided that the tape is pliable and can be used with a suitable adhesive. The thickness can be varied provided that the tape remains pliable.

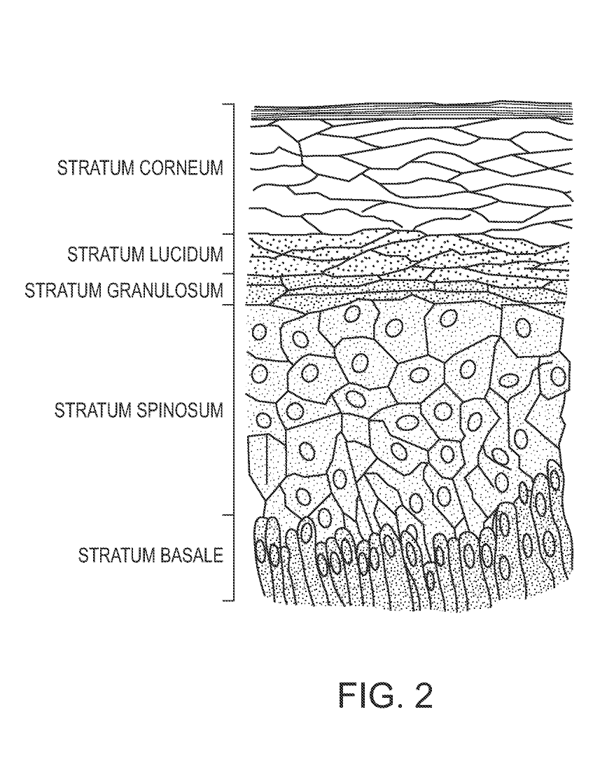

The term “skin” refers to the external dermal layer composed of three primary layers: the epidermis, which provides waterproofing and serves as a barrier to infection; the dermis, which serves as a location for the appendages of skin; and the hypodermis (subcutaneous adipose layer). The epidermis is the outermost layer of the skin, composed of terminally differentiated stratified squamous epithelium, acting as the body's major barrier against an inhospitable environment. The epidermis is composed of 4-5 layers depending on the region of skin being considered. Those layers in descending order are the stratum corneum, stratum lucidum, stratum granulosum, stratum spinosum, and stratum basale. The term Malpighian layer refers to both the basal and spinosum layers. See FIG 2.

The term “sulfur mustard” refers to bis(2-chloroethyl) sulfide; 2,2′-dichloroethyl sulfide. The NATO military designation for mustard is H or HD (H contains about 20% impurities while HD is a distilled material about 98% pure). The name “mustard” is believed to come from its reported smell and taste and from its yellow color. It is an oily liquid of low volatility with a molecular weight of 159.09, specific gravity of 1.27, a vapor density of 5.4 (relative to air equaling 1), and a freezing point of 57 degrees Fahrenheit.

A “vesicant” is a substance, whether animal, plant or synthetic, that causes vesicles, or blisters to include naturally occurring substances such as poison ivy, or synthetic chemicals.

### Antibodies

Suitable antibodies that may be employed within the scope of the method of the invention herein include those antibodies specific to keratin adducts formed following HD exposure, as well as those polyclonal antibodies having specificity to the total global pathology following HD exposure. Reference to polyclonal antibodies to HD exposure is intended to refer to those antibodies that are sensitive to changes occurring to, for example, all skin cells that have been exposed to sulfur mustard—including, but not limited to, changes to the keratin cells. Stated differently, they are specific to keratin adduct, as well as other adducts (other skin cell adducts—i.e., DNA, cell membrane, mitochondria, etc.) formed after exposure to HD. They are useful to identify the total cytopathology in HD exposed skin Applicants contend that the antibodies described herein as having specificity to the global cytopathology of HD skin exposure are novel.

### Tapes

The types of tape that may be employed within the scope of the present invention include, but are not limited to, dermal tapes approved for medical use. These tapes are to be transparent so as to be suitable to identify cells thereon via optical analysis, and double backed (double sided—containing adhesive on both sides). The tape is to have medical adhesive on at least one of its two surfaces. The side of the tape having medical adhesive thereon is the side suitable for use against the skin surface to be evaluated herein. The adhesive on one side of the tape facilitates its adherence to a slide suitable for optical viewing via a microscope or like optical device. The other adhesive surface is used to collect a sample to be evaluated. In use, one side of the tape may be affixed to a slide, while the backing on the other side of the tape may be left intact until its use is needed for sample collection. A suitable tape, by way of non-limiting illustration only, is the transparent polyethene, 6.3 mil double coated medical tape, with #80 liner sold by the product number 1522 by 3M™ Corporation (Minnesota, Minn.).

In collecting a sample in order to determine whether a subject has been exposed to HD, the backing of the suitable transparent tape is removed and the adhesive of the now exposed adhesive of the tape placed against the surface of the subject to be evaluated (i.e., the skin) so as to obtain a sample of the surface thereon. Immunohistochemical procedure, such as indirect immunoperoxidase procedure, using an antibody of the type described herein is then performed on the sample collected and present on the tape. The treated sample present on the tape is then examined using an optical device to determine the presence or absence of HD-adducted pathology therein.

Suitable optical devices that may be employed herein include a scientific microscope, such as a field microscope, and the like.

The invention herein is not limited to a method for determining whether a subject has been exposed to HD, a kit for said use or novel antibodies useful for this identified purpose. The invention may be modified for application in determining the exposure of a subject to other chemical toxicants or biological toxins such as, but not limited to, bacteria (i.e., staphylococci), fungi (i.e., candidiasis), virus (i.e., herpes), and other inflammation causing agents/cells (i.e., neutrophils), etc. It is intended that these modifications are within the scope of the present invention.

The following examples illustrate the characterization and uses of the various embodiments of the invention. These examples are illustrative only and do not limit the scope of the invention.

### EXAMPLE 1

### Materials and Methods

### Human Skin Tape Stripping

Double-sided optically clear tapes, Y and Z, and medical grade double-sided tape, A, were purchased from Light Fabrications, Inc. (Rochester, N.Y.). Tapes were cut into one-centimeter squares and adhered to 1′×3′ microscope glass slides; The volar surface of forearms of willing investigators were cleaned with sterile alcohol pads and air dried. Backing from slide tapes was removed and the whole slide pressed. Firmly on the forearm for about 10 seconds. Slides were carefully removed at a 45-degree angle to ensure even cell adherence to the tape's adhesive surface. The slides with attached cells were subjected to different experimental staining and handling paradigms identified below. After each experiment, slides were cover-slipped with permount mounting media and. A 24 mm×30 mm cover glass. Slides were dried overnight and photomicrographed using an OLYMPUS VANOX™ (Olympus Imaging America, Inc., Center Valley, Pa.) light microscope fitted with a NIKON DIGITAL SIGHT™ camera (Nikon, Inc, Melville, N.Y.). Finalized pictures were adjusted by ADOBE PHOTOSHOP ELEMENTS™ (Adobe, Inc., San Jose, Calif.), using white point level adjusters.

### Nonspecific Staining

To determine a broad spectrum of effects, all tapes were subjected to six different staining protocols for differing time periods with and without hotplate heating. Staining was performed with either sequential applications of staining ingredients or with premixed solutions of staining ingredients: 1) 100% methanol pretreatment+sequential stain application, 2) 100% methanol pretreatment+premixed stain application, 3) 100% acetone pretreatment and sequential stain application, 4) 100% acetone pretreatment and premixed stain application, 5) 1% TRITON•X-100™ (Dow Chemical, Midland, Mich.) and sequential stain application, 6) 1% TRITON X•-100™ and premixed stain application. Pretreatments with methanol, acetone and TRITON X-100 were conducted for 10 minutes at room temperature followed by a rinse in MILLIPORE™ (Billerica, Mass.) deionized water. Control slides received phosphate buffered saline (PBS) pretreatment and sequential stain or premixed stain application. For all sequential stain applications a 1:1 mixture of methylene blue/azure II was applied to the slide for 30 seconds, rinsed with deionized water and air dried. After drying, a 2:1 ratio of sodium borate and basic fuchsin was applied for 30 seconds followed by a rinse with MILLIPORE™ deionized water and air drying. All solutions were dispensed through a 0.22 μm MILLIPORE™ filter affixed to a 10 cc syringe. For all premixed applications, one part methylene blue, one part azure II, one part basic fuchsin and two parts sodium borate were premixed and dispensed through a 10 cc syringe. This stain mixture was applied to tapes for one minute then rinsed with MILLIPORE™ deionized water, air dried and cover-slipped. Selected slides were heated during staining procedures by placing the slide on a hotplate for 30 seconds.

### Results

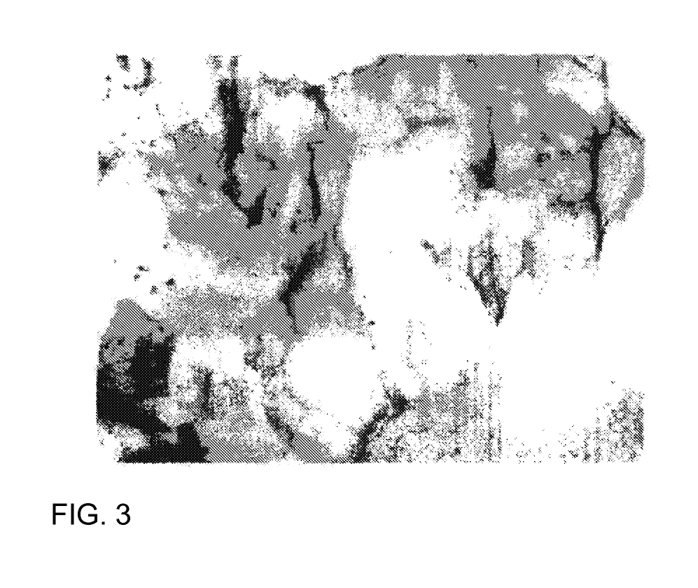

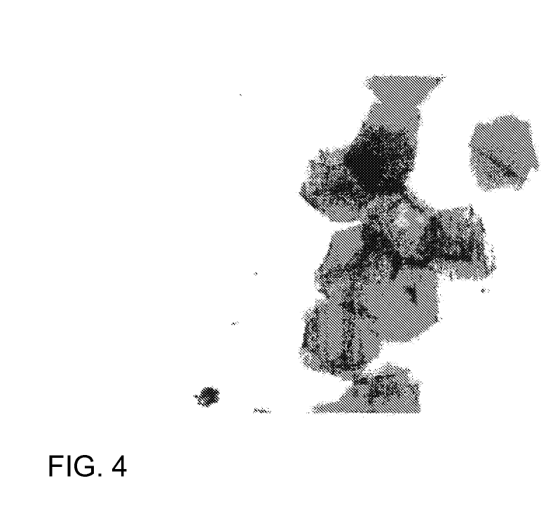

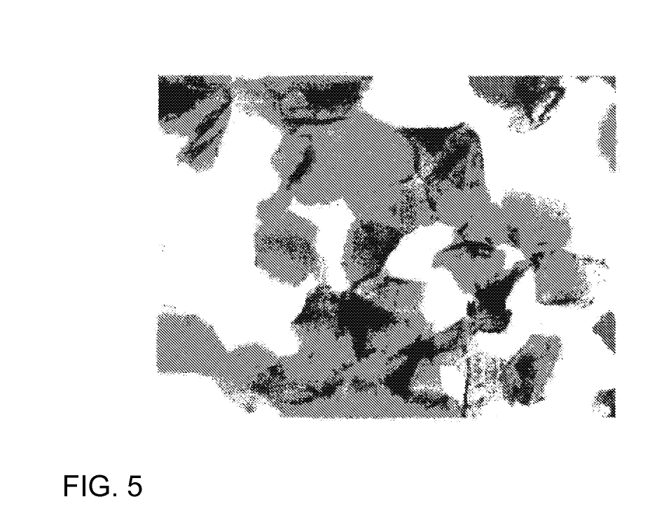

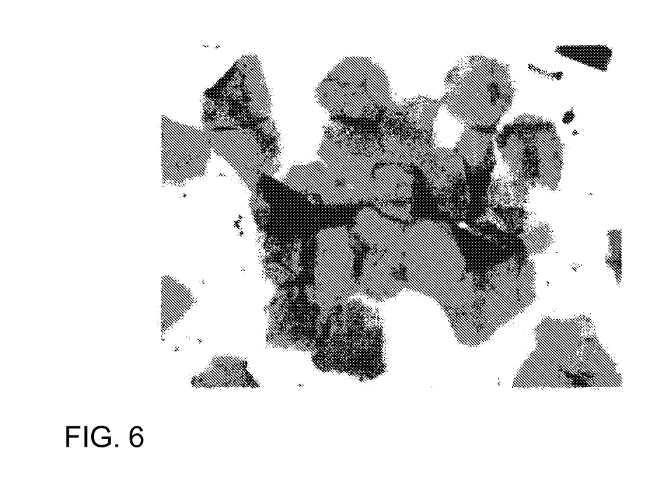

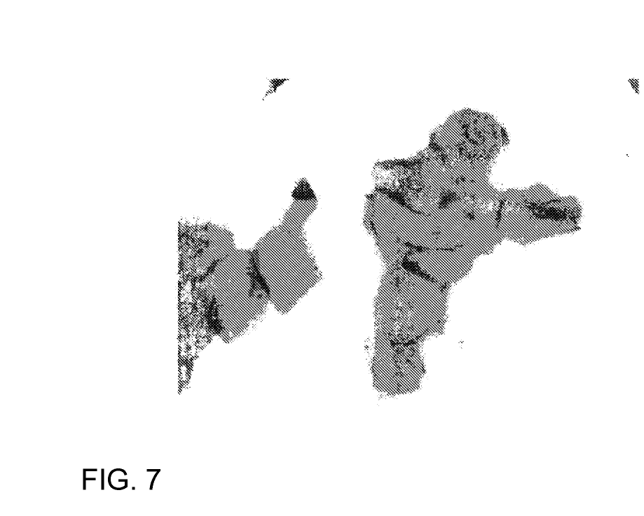

Medical grade double-backed tape, A, stood up poorly to all procedures (see FIG. 3). Backgrounds were heavily stained, and adhesion between tape and cell seemed to be lost after pretreatment. Many of the remaining cells on tape A were folded, creating deep dye pockets. On the other hand, optically clear tapes Y and Z presented clean, clear backgrounds with vividly dyed cells (see FIGS. 4-7). The integrity of adhesion between the tape and the cells seemed undiminished after pretreatments. Cells appeared flat with well demarcated edges. Most staining procedures produced vivid blue stains with consistently clear backgrounds. Methanol• and TRITON X-100™ pretreated tapes appeared more uniformly dyed than those pretreated with acetone and PBS, while cells pretreated with TRITON X-100™ were consistently superior to those with methanol pretreatment. In all cases, the use of a hotplate presented more uniform vivid staining and consistent morphological presentations. Results with basic fuchsin in staining sequences were inconsistent.

Medical grade double coated tapes suitable for use in the various embodiments of this invention are manufactured by 3M Inc.™ according to the parameters described in Table A.

TABLE A

| | | Polyethylene Carrier | | | | | |
|---|---|---|---|---|---|---|---|
| Properties | | 1509 | 1510 | 1517 | 1522 | 9874 | 9889 |
| Sterilization | | G, E | G, E | G, E | G, E | G, E | G, E |
| Hypoallergenic | | Yes | No | No | Yes | Yes | Yes |
| Repeat Skin Contact | | No | No | No | Yes | Yes | No |
| Comfortable | | M | H | H | M | M | M |
| Fluid Resistance | | Yes | Yes | Yes | Yes | Yes | Yes |
| Tensile | lbs/inch | 4.5 | 1.8 | 1.8 | 4.5 | 4.5 | 4.5 |
| | N/25 mm | 20 | 8 | 8 | 20 | 20 | 20 |
| Elongation | % | 200 | 800 | 800 | 200 | 200 | 200 |
| Adhesion | oz/inch | 54 | 45 LDPE | 30 LDPE | 25 | 19 | 53 |
| | N/25 mm | 14.7 | 12.5 | 8.3 | 7 | 5.3 | 14.7 |
| Caliper | mil | 3.0 | 1.0 | 1.0 | 3.0 | 3.0 | 3.0 |
| | millimeters | 0.08 | 0.03 | 0.03 | 0.08 | 0.08 | 0.08 |
| Adhesive | mil | 0.95 | 1.4 | 4.7 | 1.65 | 0.95 | 0.95 |
| coating | | | | | | | |
| Carrier | mil | 3.0 | 1.0 | 1.0 | 3.0 | 3.0 | 3.0 |
| Caliper | | | | | | | |
| Liner | mil | 4.5 | 3.2 | 4.4 | 4.5 | 3.6 | 3.5 |
| Caliper | | | | | | | |
| Liner | Lbs/ream | 80 | 54 | 58 | 80 | 60 | 60 |
| Weight | | | | | | | |
| Max | N/25 mm | 0.49 | 0.39 | 0.49 | 0.29 | 0.29 | 0.49 |
| Release | | | | | | | |
| Max | cm | 122 | 69 | 69 | 122 | 122 | 122 |
| Width | | | | | | | |
| Max | meters | 366 | 329 | 329 | 366 | 457 | 366 |
| Length | | | | | | | |
| Roll | cm | 36 | 34 | 34 | 37 | 34 | 37 |
| Diameter | | | | | | | |
| Shelf Life | Years | 2 | 2 | 2 | 3 | 2 | 2 |

### DISCUSSION AND CONCLUSION

The results of this morphological technical study demonstrate that tapes Y and Z are superior to tape A for nonspecific staining, microscopic clarity and morphologic integrity. Tape Y is less expensive and more readily available from the manufacturer than tape Z. making tape Y the more practical tape of choice for these studies. Permeabilization and fixation pretreatment experiments with tape Y show that 100% methanol and 1% TRITON X-100™ are superior to 100% acetone and PBS for cellular morphological and staining presentations with little effect on cell adhesion, tape integrity, or image capturing. At this time, based upon series of experiments the tape of choice for planned subsequent noninvasive immunodiagnostic/confirmatory study of HD skin exposure is optically clear tape Y with either 100% methanol or 1% TRITON X-100 as pretreatment. As expected most cells adhering to the tapes were cells of the stratum corneum with occasional cells of the stratum granulosum as recognized by cytoplasmic keratohyaline granules.

### EXAMPLE 2

Although used for there is not a uniform approach for performing sampling of skin stripping new instrumental procedures are available that provide the means of measuring the physical condition of the skin or for quantifying exogenous or endogenous compounds present within the skin.

The results of adhesive tape stripping samples were compared using three types of analysis: protein assay of keratin mass and two instrumental approaches using light reflection and pixilation of the digitized image (CUDERM™ Bionet, Inc. Spring, Tex. and VISIOSCAN VC 98™, COURAGE™ and KHAZAKA Koln, Germany).

### Methods and Materials

Adhesive tape strip samples were collected using a standardized technique from paired sites on the palm, wrist and dorsal forearm. One sample was analyzed using the VISIOSCAN VC 98™ for skin flakes that provided total area covered and a calculated desquamation index (D.I.) based on the formula by Schatz et al. [32]. These samples were subsequently analyzed for total protein using a human keratin standard (SIGMA™, St. Louis, Mo.) and a modified Bradford assay (AMRESCO™, Solon, Ohio). The other paired sample was analyzed by CUDERM/BIONET™ for skin flakes and a D.I. At each skin, 10 samples were taken to determine how depth of sampling might influence the results. Sample sets were collected from three volunteers who had normal skin except for 10 random from one person with chronic eczema. Samples were collected during summer months.

### Results

112 tape strip tapes were analyzed by the 3 techniques. Most of the BIONET™ results produced D.I.'s of less than 1 when the skin is considered “normal”. The blank-corrected protein analysis also produced measurable results for most samples. Regarding the VISIOSCAN™, by adjusting the pixel threshold levels a D.I. could be generated for all samples. There was no correlation between the VISIOSCAN™ D.I. values and protein unless the protein mass was above 500 μg/sample. Similar results were found using the CUDERM/BIONET™ analysis. The stratum corneum mass removed at three skin sites using protein analysis indicates substantially more removal In the first three samples than in the last 7 samples, which corresponds to most other previous studies.

### Discussion

The data suggest that when lower amounts of stratum corneum, as is typical from healthy skin is sampled with adhesive tape, neither of the digitized light reflection imaging methods are capable of discerning the difference up to 500 μg/sample or ˜130 μg/cm². Above this amount the correlations of the VISIOSCAN™ and BIONET™ desquamation indices and protein were linear with r²=0.88 and 0.40 respectively. Therefore, using light reflection imaging is not likely to be very useful for characterizing the mass of stratum corneum adhering to adhesive tapes when it is desired to normalize the analytical results for exogenous compounds that are sampled by the total sampled mass. These results also suggest that the light reflection imaging methods are insensitive to minor degrees of skin flakiness among healthy skin. Presently, it is not known if any modifications to these techniques might improve their sensitivity. The consecutive tape strip sampling data correspond to most findings in that the first few tape strips remove appreciably more stratum corneum than the latter ones. Some of the earlier studies which used gravimetric techniques may have been biased if the was not washed first and the superficial sebum other lipids not removed, but we believe that the present protein assay would be minimally biased by this contamination.

### CONCLUSIONS

This study suggests that light reflection imaging approaches are not sensitive to accurately detect the amounts of stratum corneum adhering to adhesive tape strips when the samples are from normal, healthy skin when compared to a protein assay. This study also suggests that appreciably higher amount of stratum corneum are removed from the first three samples of the wrist ad volar forearm but not palm.

### EXAMPLE 3

### Approach 1: Immunization with Keratinaocyte Whole Cell Lysate

Mustard Gas exposed Keratinocytes Protein Preparation:

Mustard gas exposed cells—after optimal exposure, wash the cells (three times using sterile PBS) to remove the media components by centrifugation. Finally, suspend the cells in sterile PBS.

—Adjust cell density to 10 million cells/Ml. Freeze the cells and ship on dry ice. Freeze thaw the cell suspension several times to rupture the cells. Both soluble and insoluble proteins keratinocyte whole cell lysate will be used for the immunization. Measure protein concentration by BCA method.

Two rabbits will be used for the immunization of keratinocytes whole cell lysate.

### Rabbit Protein Protocol, 118 Day:

TABLE B

| Day: | Procedure: |
|---|---|
| 0 | NZW or Elite NZW Female Rabbit Pre-bleed |
| | (Avg. 5 ml serum) |
| | 10 ID/SC: 250 μg of protein with FCA |
| 21 | Boost SC: 125 μg with FIA |
| 31 | Test Bleed (Avg. 5 ml serum) |
| 32-38 | ELISA Titer Assay of Bleed* |
| 42 | Boost SC: 125 μg with FIA |
| 52 | Test Bleed (Avg. 5 ml serum) |
| 63 | Boost SC: 125 μg with FIA |
| 73 | Production Bleed (Avg. 20. MI serum) |
| 84 | Boost SC: 125 μg with FIA |
| 94 | Production Bleed (Avg. 20. MI serum) |
| 95-101 | ELISA Titer Assay of Bleed* |
| 105 | Boost SC: 125 μg with FIA |
| 112 | Production Bleed (Avg. 20. MI serum) |
| 118 | Terminal Bleed (Avg. 50 ml serum) |

Total material required for injection for the above program is 875 μg/animal. Total amount of serum expected from a rabbit on the above protocol is approximately 120 Mi.

It is recommended that a minimum of two rabbits be used due to biological variations. Immunogen is emulsified in Freund's Complete Adjuvant (FCA) for initial injections. Freund's Incomplete Adjuvant (FIA) is used for all subsequent injections (boosts). Projects follow a three-week cycle of boosts. Test bleeds are taken approximately 10 days after the boosts.

### *Optional ELISA:

We suggest testing the first bleed for the presence of antibodies against the injected imnumogen. Information gained from an ELISA at this point will provide an opportunity for you to make adjustments to immunization schedule. Additional ELISA's performed later in the project will give you valuable information that can be used to determine which sera samples to include in your research or which bleeds you would like to have purified.

### Affinity Purification

The antibody of interest can be purified away from a complex mixture of biological samples by removing specific contaminants from a sample containing a protein of interest by negative selection. Using normal keratinocytes lysate to prepare the column. In this negative selection, the undesired or antibodies produced to non-Alkylated keratinocyte protein will be removed.

Affinity purification is potentially a very powerful tool. Negative affinity purification can generally be safely applied since the desired antibodies remain unbound and are recovered in the pool of the column pass-through and wash. The principle risk comes with the need to concentrate and buffer exchange the unbound material. For this reason, it is generally considered prudent to check the level of recovery by first purifying a small sample, 10 to 20%, of the pool intended for purification by performing a safety net.

In the Safety Net protocol, we first prepare the affinity resin(s), and purify 10 ml to 20 ml of serum to establish an optimized condition for bulk purification and provide opportunity to evaluate purified antibody in their functional assay.

### Antigen Requirements

Approximately 5-10 mg of protein is required from Normal Keratinocytes. Approximately 5-10 mg of protein is required from Mustard gas exposed Keratinocytes.

### Characterization of Antibodies

Antibodies will be characterized by: ELISA (endpoint): To determine specificity and titer; Western Blot: To determine the specificity by testing against both normal keratinocytes and mustard gas treated keratinocytes.

Approach 2: Immunizing with a Peptide

### Peptide Synthesis

A string of 11 or 12 alkylated amino acid peptides derived from exposure to sulfur mustard. These alkylated amino acids will be included in the synthesized peptide. The synthetic peptide sequence is then conjugate to keyhole limpet hemocyanin (KLH) as a vaccine carrier protein. Perform immunization of two additional rabbits using the protocols as described in approach 1.

References cited and/or included herein are incorporated by reference in their entirely.

While particular embodiments of the present invention have been shown and described, it will be obvious to those skilled in the art that changes and modifications may be made without departing from this invention. Therefore, it is intended that the claims herein are to include all such obvious changes and modifications as fall within the true spirit and scope of this invention. While a specific embodiment of the invention has been shown and described in detail to illustrate the application of the principles of the invention, it will be understood that the

### EXAMPLE 4

### Research Design and Methodology

In-vitro models (cultured human skin explants, cultured organotypic human skin) and the in-vivo model (HGP) skin sites was exposed to 10 microliter HD sulfur mustard by vapor cup for 8 minutes. At post-exposure times of 2h, 4h, 8h, and 12h, medically approved double back transparent dermal tape, to be affixed to a 1″×3″ microscope slide is used to strip off superficial skin cells of the stratum corneum of the exposed models. Unexposed models similarly tape-stripped are used as controls. Tapes with adherent cells are nonspecifically stained with cresyl violet or hematoxylin and eosin for microscopic identification of cell types. Replicate tapes with unstained adherent cells are immunohistochemically processed for HD-keratin adduct immunoreactivity according to the following procedure: glass-slide mounted tapes with identified adherent cells are fixed in 70% ethanol for 2 minutes, washed in phosphate-buffered saline (PBS, pH 7.6, 300 mOsm, 5 min.) and then stained with the following antibody sequence: blocking serum (3% goat serum, 20 minutes a room temperature); primary IH 10 monoclonal antibody (1:2 in PBS, overnight at 4 degrees centigrade); peroxidase-conjugated goat anti-mouse (1:1, in PBS, 2 hours at 37 degrees centigrade); diaminobenzidine (DAB, 1.5% hydrogen peroxide in PBS for 5 minutes or until reaction is visible), wash in 3 changes of MILLIPORE™ water, air dry, coverslip slides and examine by light microscopy. Immunoreactive cells will stain brown (DAB). Unreactive cels will be unstained. Tape peels of unexposed skin sites are similarly processed and used as unstained controls. Additional controls in the form of appropriate positive controls may also be implemented by utilizing non-alkylating blistering agents as test compounds.

### EXAMPLE 5

### Effective Procedure for Field Kit Adaptability

Field kit contains indirect immunoperoxidase lyophilized primary antibody, secondary antibody, avidin-biotin complex and diaminobenzidine chromogen tablets. The kit houses positively charged 1″×3″ microscope slides affixed with double back medical adhesive transparent tape (2×2 cm); glass cover slips, mounting media and nonspecific staining solutions. Included with the kit is a battery operated light microscope. The field kit casing is fitted with adjusting padded dividers and a lid organizer. The field kit case is further designed to accommodate all reagents, dispensers, slides and a light microscope for field use.

Appropriate light microscopes include commercially available battery-operated LED light sourced light microscopes such as the EVOLUTION™ hand held microscope sold by PYSER-SGI (Kent, United Kingdom).

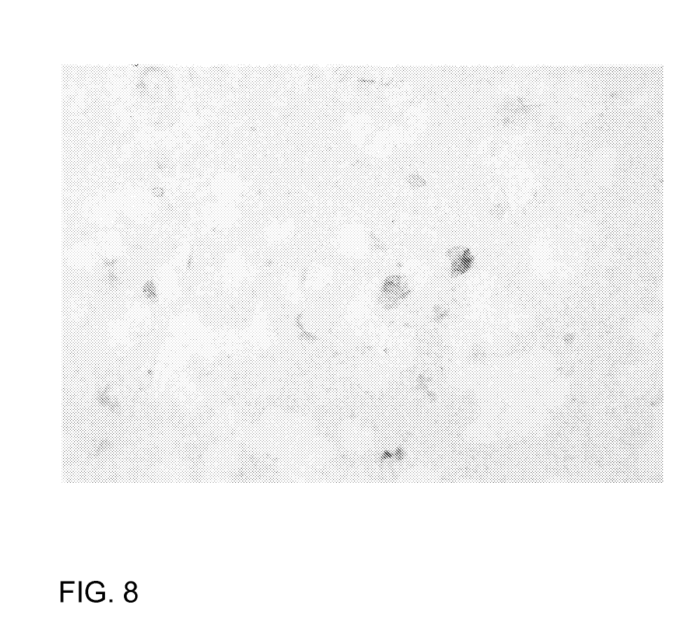

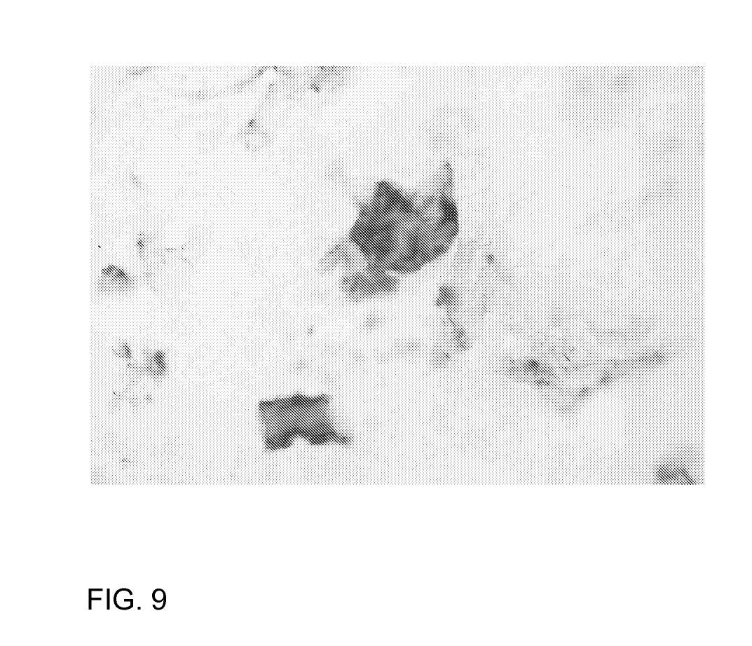

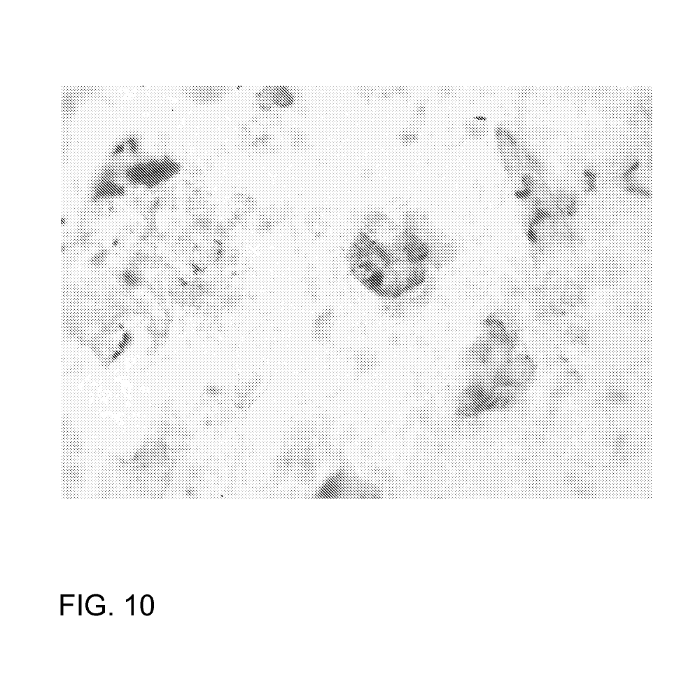

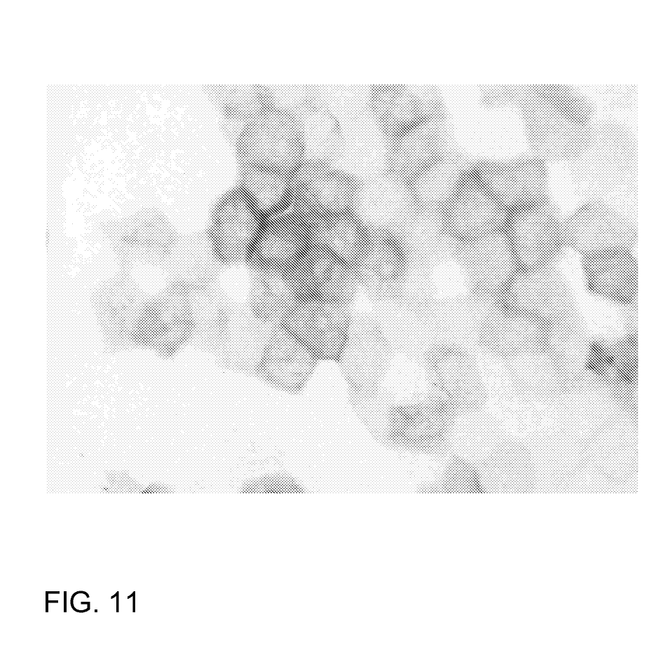

FIGS. 8-11, illustrate on-the-tape skin cells immunoreacted to determine the presence of keratin adducts formed following HD exposure. These are representative of on-the-tape immunostaining of skin peels of skin exposed/unexposed to HD. The antibody used herein is specific to keratin adducts.

Various features of novelty that characterize the invention are pointed out with particularity in the claims annexed to and forming a part of this disclosure. For a better understanding of the invention, its operating advantages and specific objects attained by its uses, reference is made to the accompanying drawings and descriptive in which a preferred embodiment of the invention is illustrated.

Numerous modifications and variations of the present invention are included in the above-identified specification and are expected to be obvious to one of skill in the art. Such modifications and alterations to the compositions and processes of the present invention are believed to be encompassed in the scope of the claims appended hereto.

### REFERENCES

The contents of each of which, and the contents of every other publication, including patent publications such as PCT International Patent Publications, being incorporated herein by this reference.
1. Marzulli, F. N., et al., Marzulli and Maibach's dermatotoxicology. 7th ed. 2008, Boca Raton: CRC Press. xxv, 1015 p.
2. Emanuel, P. A., et al., Recombinant antibodies: a new reagent for biological agent detection. Biosens Bioelectron, 2000. 14(10-11): p. 751-9.
3. Petrenko, V. A. and V. J. Vodyanoy, Phage display for detection of biological threat agents. J Microbiol Methods, 2003. 53(2): p.253-62.
4. Bikker, F. J., et al., Detection of sulfur mustard adducts in human callus by phage antibodies. Chem Biol Drug Des, 2007. 69(5): p. 314-20.
5. Parmley, S. F. and G. P. Smith, Antibody-selectable filamentous fd phage vectors: affinity purification of target genes. Gene, 1988. 73(2): p. 305-18.
6. Hogrefe, H. H., et al., Cloning in a bacteriophage lambda vector for the display of binding proteins on filamentous phage. Gene, 1993. 137(1): p. 85-91.
7. Dooley, H., et al., Stabilization of antibody fragments in adverse environments. Biotechnol Appl Biochem, 1998. 28 (Pt 1): p. 77-83.
8. Hoess, R. H., Protein design and phage display. Chem Rev, 2001. 101(10): p. 3205-18.
9. Skerra, A., Alternative non-antibody scaffolds for molecular recognition. Curr Opin Biotechnol, 2007. 18(4): p. 295-304.
10. Noort, D., H. P. Benschop, and R. M. Black, Biomonitoring of exposure to chemical warfare agents: a review. Toxicol Appl Pharmacol, 2002. 184(2): p.116-26.
11. Fidder, A., et al., N7-(2-hydroxyethylthioethyl)-guanine: a novel urinary metabolite following exposure to sulphur mustard. Arch Toxicol, 1996. 70(12): p. 854-5.
12. Read, R. W. and R. M. Black, Analysis of beta-lyase metabolites of sulfur mustard in urine by electrospray liquid chromatography-tandem mass spectrometry. J Anal Toxicol, 2004. 28(5): p. 346-51.
13. Riches, J., R. W. Read, and R. M. Black, Analysis of the sulphur mustard metabolites thiodiglycol and thiodiglycol sulphoxide in urine using isotope-dilution gas chromatography-ion trap tandem mass spectrometry. J Chromatogr B Analyt Technol Biomed Life Sci, 2007. 845(1): p.114-20.
14. van der Schans, G. P., et al., Standard operating procedure for immunuslotblot assay for analysis of DNA/sulfur mustard adducts in human blood and skin. J Anal Toxicol, 2004. 28(5): p. 316-9.
15. van der Schans, G. P., et al., Immunochemical detection of sulfur mustard adducts with keratins in the stratum corneum of human skin. Chem Res Toxicol, 2002. 15(1): p. 21-5.
16. Noort, D., et al., Alkylation of human serum albumin by sulfur mustard in vitro and in vivo: mass spectrometric analysis of a cysteine adduct as a sensitive biomarker of exposure. Chem Res Toxicol, 1999. 12(8): p. 715-21.
17. Noort, D., et al., Diagnosis and dosimetry of exposure to sulfur mustard: development of a standard operating procedure for mass spectrometric analysis of haemoglobin adducts: exploratory research on albumin and keratin adducts. J Appl Toxicol, 2000. 20 Suppl 1: p. S187-92.
18. Fidder, A., et al., Synthesis, characterization, and quantitation of the major adducts formed between sulfur mustard and DNA of calf thymus and human blood. Chem Res Toxicol, 1994. 7(2): p.199-204.
19. Griffiths, A. D., et al., Isolation of high affinity human antibodies directly from large synthetic repertoires. EMBO J, 1994. 13(14): p. 3245-60.
20. Winter, G., et al., Making antibodies by phage display technology. Annu Rev Immunol, 1994. 12: p.433-55.
21. Porter, D. e.a., A new method for measuring replacement of epidermis and stratum corneum in human skin. Journal of Investigative Dermatology, 1967. 49: p. 251-255.
22. Jenkins, H. L. e.a., An adhesive-tape stripping technique for epidermal histology. Journal of Society of Cosmetic Chemists, 1969. 20: p. 451-466.
23. Rougier, A., et al., The measurement of the stratum corneum reservoir. A predictive method for in vivo percutaneous absorption studies: influence of application time. J Invest Dermatol, 1985. 84(1): p. 66-8.
24. Rougier, A., et al., In vivo correlation between stratum corneum reservoir function and percutaneous absorption. J Invest Dermatol, 1983. 81(3): p. 275-8.
25. Rougier, A., et al., Regional variation in percutaneous absorption in man: measurement by the stripping method. Arch Dermatol Res, 1986. 278(6): p. 465-9.
26. Dupuis, D., et al., In vivo relationship between horny layer reservoir effect and percutaneous absorption in human and rat. J Invest Dermatol, 1984. 82(4): p. 353-6.
27. Dupuis, D., et al., The measurement of the stratum corneum reservoir: a simple method to predict the influence of vehicles on in vivo percutaneous absorption. Br J Dermatol, 1986. 115(2): p. 233-8.
28. Serup, J., A. Winther, and C. Blichmann, A simple method for the study of scale pattern and effects of a moisturizer—qualitative and quantitative evaluation by D-Squame tape compared with parameters of epidermal hydration. Clin Exp Dermatol, 1989. 14(4): p. 277-82.
29. Cullander, C., P. G. Grant, and G. Bench, Development of a low-metal adhesive tape to detect and localize metals in or on the stratum corneum at parts per million levels. Skin Pharmacol Appl Skin Physiol, 2001. 14 Suppl 1: p. 46-51.
30. Cullander, C., et al., A quantitative minimally invasive assay for the detection of metals in the stratum corneum. J Pharm Biomed Anal, 2000. 22(2): p. 265-79.
31. Nylander-French, L. A., A tape-stripping method for measuring dermal exposure to multifunctional acrylates. Ann Occup Hyg, 2000. 44(8): p.645-51.
32. Schatz, H. e.a., Quantification of dry (xerotc) by image analysis of scales removed by adhesive discs (D-Squames). J. Soc. Cosmet. Chem., 1993. 44: p. 53-63.

What is claimed is:

1. A polyclonal antibody to sulfur mustard adducts purified from mammalian antisera, said antisera derived from the protocol described in Table B herein using mammals immunized against said sulfur mustard adducts.

2. The polyclonal antibody of claim 1, wherein said sulfur mustard adducts are derived from keratinocytes whole cell lysates.

3. A non-invasive method for determining a subject's dermal exposure to an agent, wherein said method comprises:
applying a transparent adhesive tape to a target area of the skin of the subject in a manner sufficient to isolate an epidermal sample adhering to the adhesive tape, wherein the epidermal sample comprises cells from the stratum corneum of the subject;
employing an immunohistochemical procedure on the sample present on said tape resulting in a treated sample, said immunoshitochemical procedure employing an antibody specific to the adducts of said agent; and
examining the treated sample present on said tape to determine the presence of said agent adduct therein.

4. The method of claim 3, said transparent tape is double sided, with adhesive on both sides of said transparent double sided tape.

5. The method of claim 3, wherein said immunohistochemical procedure is indirect immunoperoxidase procedure.

6. The method of claim 3, wherein said antibody is a polyclonal antibody.

7. The method of claim 3, wherein said antibody is a monoclonal antibody.

8. The method of claim 3, wherein said antibody is a recombinant antibody.

9. The method of claim 3, wherein said antibody is a phage antibody.

10. The method of claim 3, wherein said antibody is a synthetic protein constructed on a non-immunoglobulin scaffold selected from the group consisting of protein A, lipocalins, fibronectin domains, ankryin consensus repeat domains and thioredoxin.

11. The method of claim 6, wherein the polyclonal antibody is purified from mammalian antisera obtained in the protocol described in Table B herein.

12. The method of claim 4, wherein the double sided transparent tape is selected from the group consisting of tapes described in Table B herein.

13. The method of claim 4, wherein the double sided transparent tape comprises a soft and pliable polyethylene backing layered with an acrylate based adhesive.

14. The method of claim 3, wherein said antibody is specific to keratin adducts formed following mustard sulfur exposure.

15. The method of claim 3, wherein said treated sample is examined using a device selected from the group consisting of a densitometer, a reflectometer, a light microscope and a calorimeter.

16. The method of claim 3, wherein said agent is selected from the group consisting of sulfur mustard, nitrogen mustard, and lewisite.

17. A kit for use in determining whether a subject has been exposed to an agent, wherein said kit comprises
at least one positively charged microscope slide;
mounting media;
glass cover slips;
nonspecific staining solutions;
double-sided transparent tape suitable to obtain a skin sample from said subject;
indirect immunoperoxidase lyophilized primary antibody specific to adducts formed by said agent;
a secondary antibody;
avidin-biotin complex;
diaminobenzidine chromogen tablets;
case designed to accommodate all said kit components.

18. The kit of claim 17, wherein said primary antibody is specific to keratin adducts formed following sulfur mustard exposure.

19. The kit of claim 17, wherein said primary antibody is a polyclonal antibody purified from mammalian antisera, said antisera derived from the protocol described in Table B herein using mammals immunized against sulfur mustard adducts.

20. The kit of claim 17, wherein said kit further comprises a device selected from the group consisting of a densitometer, a reflectometer, a light microscope and a calorimeter.

21. The kit of claim 17, wherein said kit further comprises instructions for use of said kit in determining whether said subject has been exposed to said agent.

22. The kit of claim 17, wherein the agent is selected from a group consisting of sulfur mustard, nitrogen mustard, and lewisite.
